(19) United States
(12) Patent Application Publication
(10) Pub. No.: US 2025/0332380 A1
(43) Pub. Date: Oct. 30, 2025

(54) HIGH PERFORMANCE BRAID-FREE MICROCATHETERS WITH IMPROVED VASCULATURE AND LESION CROSSABILITY CHARACTERISTICS AND RESPONSE

(71) Applicant: CARDIOVASCULAR SYSTEMS, INC., St. Paul, MN (US)

(72) Inventors: Jeffrey A. McBroom, Champlin, MN (US); Craig Stowell, Watertown, MN (US); Jonathan Wong, Columbia Heights, MN (US); Bruce H. Asmus, Minnetonka, MN (US)

(21) Appl. No.: 18/870,167

(22) PCT Filed: Jun. 2, 2023
(86) PCT No.: PCT/US2023/067829

§ 371 Date: Nov. 27, 2024

Related U.S. Application Data
(60) Provisional application No. 63/365,715, filed on Jun. 2, 2022.

(51) Int. Cl. A61M 25/00; A61M 39/02

(52) U.S. Cl. CPC A61M 25/0053; A61M 39/0247

(57) ABSTRACT

Embodiments of the disclosed microcatheters comprise an inner tube that extends from a distal tip to a proximal hub. One embodiment of a microcatheter comprises a first inner coil wound around a length of the inner tube, a second middle coil wound around the first coil and in a different winding direction or lay than the winding direction or lay of the first coil, and a third outer coil wound around a proximal portion of the second coil and in a different winding direction or lay than the winding direction or lay of the second coil. In one disclosed embodiment, the first, second and third coils include distal ends that terminate distally together at a common location that is spaced proximally from the distal tip. Gaps in one or more of the first, second or third coils may be provided between groups or sections of wire filars forming the coils to improve flexibility while maintaining sufficient axial force transmission and torque capabilities.

### CROSS-REFERENCE TO RELATED APPLICATIONS

This application claims the benefit of provisional application Ser. No. 63/365,715 filed Jun. 2, 2022 and titled HIGH PERFORMANCE MICROCATHETERS, the entire content of which is incorporated herein by reference.

### STATEMENT REGARDING FEDERALLY SPONSORED RESEARCH OR DEVELOPMENT

Not Applicable

### TECHNICAL FIELD

The disclosure is related to intravascular access using microcatheters.

### BACKGROUND

Catheters are medical devices that include a lumen for passage of fluids and/or devices such as guidewires. The art is replete with catheters used for a variety of medical purposes. Examples include U.S. Pat. Nos. 7,981,091; 9,636,477; 9,782,561; 10,065,331; 10,166,363; 10,238,834; 10,258,767; 10,493,234; 10,835,283 and 10,912,921.

Microcatheters are typically catheters with an outer diameter of less than about 1.25 mm, with most microcatheters comprising a diameter of less than about 1.0 mm. Some microcatheters are not called upon for rigorous performance characteristics and tend to be inexpensively constructed. Other microcatheters are required to traverse challenging, labyrinth-like vessels within less than healthy patients. Such catheters may present a challenge to construct and in some instances, some performance characteristics may be sacrificed in favor of others.

Some microcatheters are designed for use in or near the brain. These devices are designed to be highly flexible and as such, would be incapable of use in applications with tortuous or semi-blocked paths. The flexibility of those catheters is useful to traverse the base of the skull, but that same flexibility renders them useless for other challenges, including many uses in the peripheral or coronary vasculature. Intravascular microcatheters for peripheral or coronary access may be designed to include a passage for a 0.014 inch guidewire.

Percutaneous intravascular procedures such as angioplasty (with or without stenting), lithoplasty, atherectomy, thrombectomy, and lithoplasty may be used to treat intravascular targets. In an exemplary case, below-the-knee (“BTK”) lesions may be treated using, e.g., angioplasty and/or atherectomy to effectively treat BTK lesions and restore blood flow and improve limb salvage potential. The technical success of any intravascular procedure to treat an exemplary lesion such as a BTK lesion initially depends on the ability to cross the target lesion. The choice of vascular access appears critical in the exemplary BTK lesion intervention. Various vascular access options are available, including radial artery access, ipsilateral femoral access, contralateral femoral access and retrograde distal access. See, e.g., Li, Y. et al., Antegrade vs crossover femoral artery access in the endovascular treatment of isolated below-the-knee lesions in patients with critical limb ischemia, Journal of Endovascular Therapy 2017; 24(3): 331-6.

Antegrade catheters may be used to reach an anatomical target of interest such as a lesion or occlusion within a blood vessel in the direction of a flow of a bodily fluid such as blood. Antegrade catheters generally must traverse a longer distance from a percutaneous access point to the target lesion, e.g., a BTK lesion, than a typical traversal distance for retrograde catheters. As a result, pushability, i.e., axial force transfer, kink resistance and torque are required performance parameters for antegrade catheters.

Retrograde catheters may be used to cross a lesion in a direction opposite to the direction of flow of a bodily fluid such as blood. There may be advantages to a retrograde crossing including that the distal, or retrograde side, of a lesion may be softer, or shaped to allow easier access, compared with the proximal or antegrade side of the lesion. Generally, retrograde microcatheters may comprise a distal profile that is smaller in diameter, with smaller crossing profile than antegrade microcatheters, and further comprise a more flexible distal profile than antegrade microcatheters which, as noted, generally require maximum pushability and torque to reach an intravascular target.

Microcatheters may be used generally to obtain collateral vessel access among other types of vessel access. In some cases microcatheters commonly used for retrograde procedures may present the best option to a physician, while a physician may prefer microcatheters commonly used for antegrade procedures in other cases. The microcatheter embodiments described herein are not intended to be limited to retrograde or antegrade.

Microcatheters include diverse performance factors and characteristics comprising one or more of at least rigidity, torque transmission; size (e.g. length, inner and outer diameters), crossing profile, flexibility, kink resistance, softness and other characteristics.

There is a need for high performance microcatheters with improved vasculature and lesion crossability characteristics and response. Some of the elements contributing to crossability include a desirable combination of small crossing profiles, an optimal flexibility range-particularly a distal region of the microcatheter and effective torque transmissibility within an optimal range, preferably a bi-directional torqueing response for at least one rotation in both clockwise and counterclockwise directions.

Embodiments of the present disclosure address these, inter alia, issues.

### SUMMARY

Embodiments of the disclosed microcatheters comprise an inner tube that extends from a distal tip to a proximal hub. One embodiment of a microcatheter comprises a first inner coil wound around a length of the inner tube, a second middle coil wound around the first coil and in a different winding direction or lay than the winding direction or lay of the first coil, and a third outer coil wound around a proximal portion of the second coil and in a different winding direction or lay than the winding direction or lay of the second coil. In one disclosed embodiment, the first, second and third coils include distal ends that terminate distally together at a common location that is spaced proximally from the distal tip. Gaps in one or more of the first, second or third coils may be provided between groups or sections of wire filars forming the coils to improve flexibility while maintaining sufficient axial force transmission and torque capabilities. An outer layer of polymer materials is provided around the coils, wherein the polymers may comprise decreasing hardness or stiffness, and increasing softness or flexibility, moving from proximal to distal along the microcatheter.

The disclosed microcatheters may be used in conjunction with a steerable guidewire to access and/or cross regions of the coronary and/or peripheral vasculature, or other vascular targets. The disclosed microcatheters may also be used to support a guidewire as it crosses a lesion, or they may be used to facilitate placement and exchange of guidewires and other interventional devices and to selectively infuse/deliver diagnostic and therapeutic agents and/or for delivery of contrast media into the coronary, peripheral, and abdominal, or other, vasculature.

The microcatheters of the present disclosure comprise embodiments of shaft constructions that provide improved crossability and other performance characteristics including, among other things, crossing profile, distal region flexibility, pushability, torque response, and kink resistance.

### BRIEF DESCRIPTION OF DRAWINGS

It is to be understood that the present invention is not limited by the embodiments described herein. Alternatively, the present invention can be used in arteries, veins, and other body vessels. By altering the size of the disclosed embodiments, the embodiments may be suitable for peripheral, coronary and neurological applications. Other features and advantages of the present invention will become more apparent from the following detailed description of the invention, when taken in conjunction with the accompanying exemplary drawings.

FIG. 1 illustrates a side view of one embodiment of the present invention;

FIG. 2 illustrates a side cutaway view of a distal region of one embodiment of the present invention.

FIG. 3 illustrates a side cutaway view of a proximal region of one embodiment of the present invention.

FIG. 4 illustrates a side cutaway view of a proximal portion of one embodiment of the present invention shown in FIG. 3.

FIG. 5 is a perspective, broken view of one embodiment of a coil assembly of the present invention.

FIG. 6 is a schematic diagram of one embodiment of the present invention.

FIG. 7 is a perspective, broken view of portions of one embodiment of a microcatheter according to the present invention with portions of an outer jacket or sleeve broken away.

FIG. 8 is an exemplary manufacturing process for one embodiment of the present invention.

FIG. 9 is a photograph of part of a test setup for torque response measurement.

FIG. 10 is a photograph of part of a test setup for torque response measurement.

FIG. 11 is a photograph of part of a test setup for flexibility testing of a distal region of a microcatheter.

FIG. 12 is a photograph of part of a test setup for flexibility testing of a distal region of a microcatheter.

### DETAILED DESCRIPTION

The following description refers to the accompanying drawings, which illustrate specific embodiments of the invention. Other embodiments having different structures and operation do not depart from the scope of the present invention.

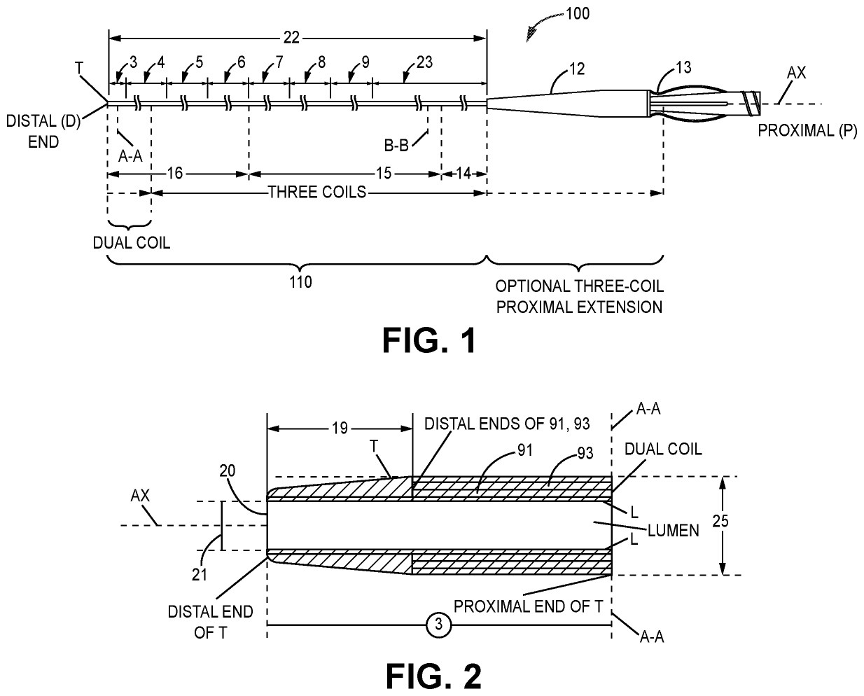

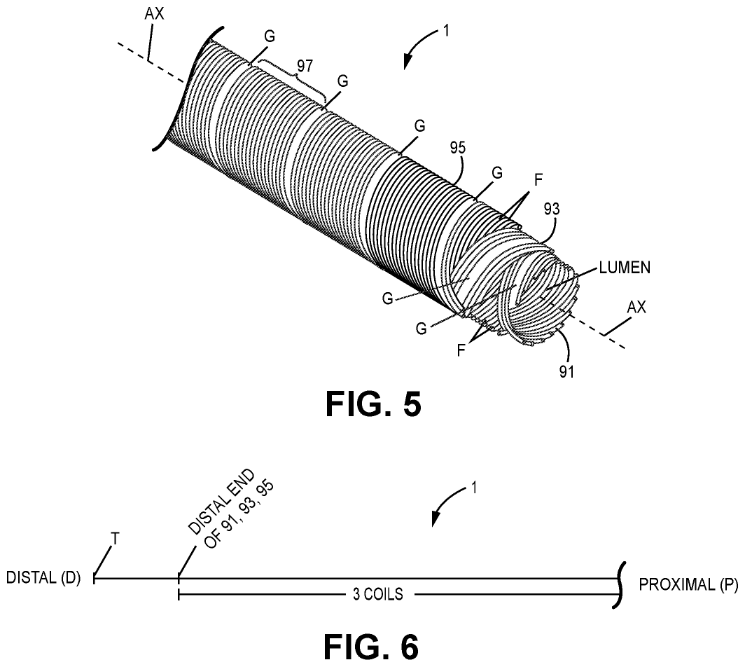

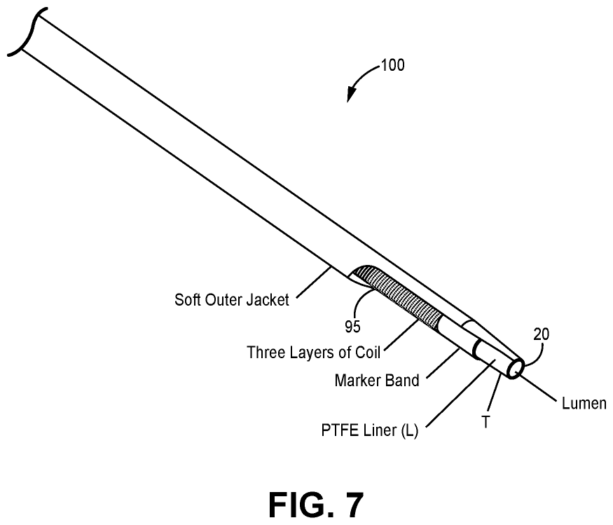

With reference generally to FIGS. 1-4, an embodiment of an exemplary microcatheter 100 is illustrated. The catheter has an elongate body 110 comprising a polymeric inner tube or liner L or coating forming at least a portion of a single inner lumen having an inner diameter 21 and an outer diameter 25 and defining a longitudinal axis AX of microcatheter 100. The elongate body 110 further comprises a proximal region 14, middle or transition region 15 and distal region 16 and a tapered distal tip T, with the smallest outer diameter at its distal tapered end which may preferably be within the range of about 0.4 mm to about 0.6 mm, though the outer diameter of the distal end of distal tip T maybe greater or less than 0.4 mm to 0.6 mm. A preferred outer diameter of the distal end of the distal tip T is approximately 0.48 mm.

As best seen in FIG. 2, the distal tip portion T has an outlet 20 of the inner lumen and an inner diameter 20. The lumen is preferably defined by a polymeric inner liner that extends along the axis AX toward the outlet 20. The liner L may be provided by any suitable material or coating such as, polytetrafluoroethylene (PTFE), silicone or another, in some embodiments lubricating, material or coating to provide a surface and/or lumen for passage of interventional devices, guidewires, infusate, drugs or the like. In one embodiment, the outlet 20 is formed when the liner L extends all the way to the outlet 20 of the distal tip T. Alternatively, the lumen may be provided by the inner portion of innermost coil 91 which, in some embodiments, may be coated with a layer of polymer or other similar material. The lumen may be suitable for passage of a 0.014 inch, or other size, guidewire.

The outer diameter 25 of the distal region 16 of the elongate body 110 is preferably less than about 1.25 mm; more preferably less than about 1.0 mm and even more preferably less than about 0.8 mm and may be larger than the smallest outer diameter of the tapered distal tip T which extends distally a distance from a distal end of the distal region 16. A particularly preferred outer diameter of distal region 16 may be approximately 0.71 mm. In certain embodiments, the crossing profile of the distal region 16 may be 2.1 F.

The microcatheter optionally includes a hub 13 operatively connected with the coil assembly 1 and/or inner liner L. The hub 13 may comprise any suitable manually graspable handle such as a 2, 3 or 4-winged hub that may include an inlet I in fluid communication with the inner liner's lumen. Alternatively, the inner liner L may extend distally along the length of the hub 13 to provide an extended lumen through hub 13. An optional strain relief 12 may be connected to the hub 13.

The distal end of the strain relief 12 may define a working length 22 of the catheter 100. The working length is preferably between about and about 115 cm and about 200 cm, more preferably between about 135 cm and about 150 cm. The strain relief 12 may be made of a material with a softer durometer than the material forming the hub 13.

The microcatheter 100 preferably includes a synthetic layer or layers surrounding the coil assembly 1. The synthetic layer or layers is depicted as including regions 3, 4, 5, 6, 7, 8, 9 and 23 but more or less discrete regions may be utilized. As best seen in FIG. 2, region 3 comprises the polymer material used to form the distal tip T. The synthetic region is preferably a polymer or an elastomer, more preferably a polymeric elastomer. Materials for the portions 4, 5, 6, 7, 8, 9, and 23 may comprise polyethylene, polyvinylpyrrolidone, polypropylene, polyethylene terephthalate, polyamide, polyester, or polyurethane, or combinations thereof. Examples include Vestamid, Pellethane, Carbothane, Nylon (e.g. Aesno 12 Nylon or Grilamid), Hytrel, Pebax or polyolefin. Preferably, the materials of portions 4, 5, 6, 7, 8, 9, and 23 do not increase in hardness and preferably decrease in durometer along the catheter's length in the direction from the proximal portion P toward the distal portion D. In one embodiment, the durometers sequentially decrease in the distal direction. The distal tip T, at region 3, may be formed from a polymer selected from the listing above and/or may comprise a material having a durometer that may be comparable to that of region 4.

Section 14 comprises an outer diameter, which may be larger than the outer diameter of section 15 which, in turn, may be larger than the outer diameter of section 16. Outer diameter differential may be achieved by providing a thicker synthetic layer in sections 14 and/or 15. In addition to providing pushability and torqueability, a larger outer diameter in at least section 16 may provide additional strain relief for the system as it may transition less abruptly with the stiffness of the strain relief 12.

In one embodiment, the outer portion of the elongate body 110 may be coated along its length with a coefficient of friction-reducing material (e.g., a hydrophilic or a hydrophobic material or combinations thereof) to facilitate insertion and trackability through vasculature.

The compositions and lengths of the polymeric portions 4, 5, 6, 7, 8, 9, and 23 are preferably diverse to impart desired structural characteristics for the catheter 100. Examples of different structures for the polymeric portions are described in Table 1 provided infra. Notably, as the skilled artisan will recognize, materials different than those disclosed in Table 1 may be used to impart the desirable features of the microcatheter 100.

The middle or transition region 15 proximally adjacent to the distal region 16 wherein the outer diameter of the middle or transition region 15 may be slightly larger than the outer diameter of distal region 16. In one embodiment, the outer diameter of the middle or transition region 15 may be preferably less than 1.1 mm, more preferably less than about 0.95 mm and more preferably less than 0.9 mm. A particularly preferred outer diameter of the middle region 15 may be 0.84 mm.

The proximal region 14 located proximally adjacent to the middle or transition region 15 and with an outer diameter than may be larger than the outer diameter of the middle or transition region 15. A preferred outer diameter of the proximal region 14 may be preferably less than 1.0 mm. A particularly preferred outer diameter of the proximal region 14 may be approximately 0.95 mm.

Generally, the outer diameter of the elongate body 110 may transition from the smallest outer diameter at the distal end of the distal tip T to the largest outer diameter at proximal region 14. When present, the transitioning outer diameter of the elongate body 110 may comprise a smoothly changing tapering outer diameter increase from distal to proximal. Stated alternatively, the outer diameter may comprise a smoothly changing decrease moving from the proximal region 14 to the distal end of the distal tip T. In other embodiments, at least part of the transition of the outer diameter of the elongate body 110 may comprise a stepped up, or gradually increasing, outer diameter moving in the proximal direction.

Accordingly, the outer diameter of the tubular portion or body 110 may remain constant or may increase, taper or step up moving in the proximal direction. The geometry of a smoothly tapering decrease in outer diameter moving in the distal direction helps to control the mechanical properties of the catheter to avoid bucking during axial loading and translation.

Generally, though the outer diameter of the tubular portion or body 110 may change along its length as described above, the inner diameter of a lumen defined by the inner tube or liner L may remain constant along its length. A preferred inner diameter of lumen may be less than about 0.55 mm. A particularly preferred inner diameter of lumen may be approximately 0.43 mm. Alternatively, in some embodiments, the inner diameter of lumen may comprise a smoothly tapering decrease moving in the distal direction.

The catheter 100 has a support assembly comprising a coil assembly 1. The illustrated embodiments do not comprise a braid, though some alternative embodiments may comprise a braid.

Referring to FIGS. 2 and 5, an exemplary coil assembly 1 comprises at least a first, innermost filar coil 91 formed of one or more filars F and wound about the axis AX in a first winding direction. The coil assembly 1 also comprises a second coil 93, formed of one or more filars F, disposed around or outside the first coil 91 and wound about the axis AX in a second winding direction different than the first wind direction. The coil assembly 1 also includes a third coil 95 formed of one or more filars F, disposed around or outside the second coil 93 and wound in a third wind direction different than the second wind direction.

With continued general reference to FIGS. 1-4, and specifically referring to FIGS. 5-6, the coil assembly 1 comprises at least a first, innermost coil 91 wound about the axis AX in a first winding direction. The coil assembly further comprises a second coil 93, surrounding at least a portion of the first coil 91 and wound about the axis AX in a second winding direction different than the first wind direction. The coil assembly 1 also includes a third coil 95 wound in a third wind direction about at least a portion of the second coil 93 and in a different wind direction than the second wind direction. In each case, as will be discussed further, the first coil 91 may be wound around the outer surface of inner liner L, the second coil 93 may be wound around the first coil 91 and the third coil 95 may be wound around the second coil 93. FIG. 5 illustrates three exemplary coils 91, 93, 95 and the different wind directions for each coil 91, 93, 95.

At least one of the coils 91, 93 and 95 may extend a different length from the proximal portion P of the catheter 100 toward the distal portion D of the catheter 100 than the remaining coils. Stated differently, the distal ends of the coils 91, 93, 95 may be proximally spaced away from the distal end of the distal tip T, wherein at least one of the proximal spacing distance(s) for the distal ends of the coils 91, 93, and/or 95 is different than the proximal spacing distance(s) for the remaining coil(s) 91, 93, 95.

As illustrated in FIG. 6, and with continued reference to FIG. 1, some embodiments of the exemplary microcatheter 100 may comprise a coil assembly 1 comprising first, second and third coils 91, 93 and 95 extending along a portion of the distal region 16 of the microcatheter 100 and terminating distally at a common location or point that is proximal to the distal end of the distal tip T. Thus, the distal ends of first, second and third coils 91, 93, 95 of the coil assembly 1 are located proximal to the distal end of the microcatheter 100 and, as illustrated, the distal end of the distal tip T.

In an alternate embodiment, two coils, e.g., 91, 93 may be provided as first coil 91 and second coil 93 are described herein, wherein the distal ends of the two coils 91, 93 are located at a common location along the inner liner L and proximal to the distal end of the microcatheter 100. In this embodiment, the first coil 91 is the inner coil surrounding at least a portion of the inner liner L and comprising a first winding direction. The second coil 93 becomes the outermost coil in this embodiment, surrounding at least a portion of the inner first coil 91 and comprising a second winding direction different from the first winding direction.

The distance between the distal end of the distal tip T and the distal ends of the first, second and third coils 91, 93, 95 of the coil assembly 1 forming a triple coil is marked as clement 19 in FIG. 2 and that distance 19 may be less than about 10 mm, more preferably less than about 5 mm and more preferably about 1 mm, though these distances are merely exemplary and other distances are within the scope of the inventions described herein.

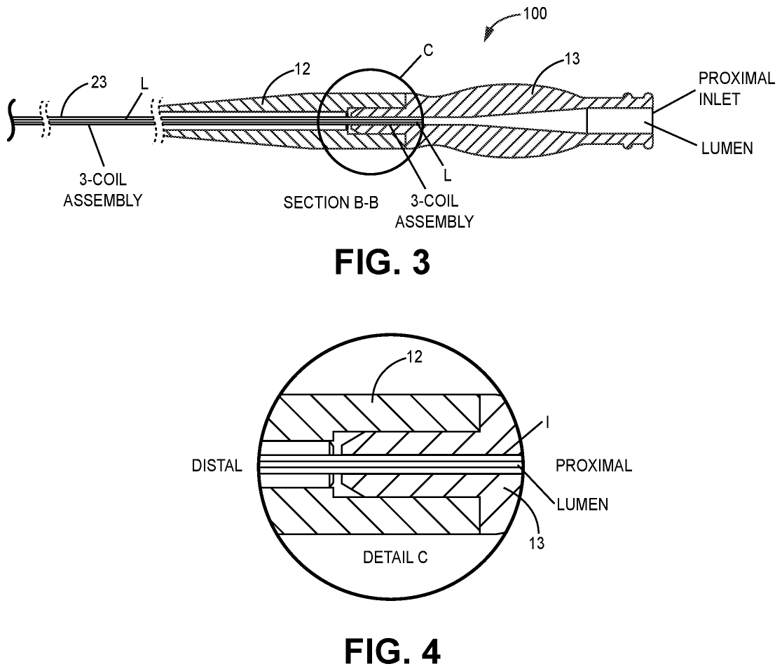

In some embodiments, the three-coil portion of the coil assembly 1 may extend proximally through strain relief element 12 and in some embodiments into the hub 13 as shown in FIG. 3.

The different winding directions of the coils 91, 93 and/or 95 provide for a microcatheter that is capable of rotating in opposing directions with substantially equal torqueing force and, therefore, provides a bi-directional rotatable microcatheter that will resist elongation and shortening during rotation in either direction. In addition, the disclosed microcatheter is capable of a plurality of rotations in one direction, e.g., clockwise or counterclockwise, with substantially equal torqueing forces produced or generated by each rotation.

As described above, the first innermost coil 91 comprises one or more filars F that are wound in an exemplary helical or spiral configuration in a first winding direction. A second middle coil 93 is formed from one or more filars F wound about the first innermost coil 91 in a second winding direction that is different from the first winding direction. Finally, a third outermost coil 95 is formed from one or more filars F that are wound about the second middle coil 93 in a third winding direction that is different from the first winding direction.

The windings in first, second and third coils 91, 93, 95 are illustrated as spiral, or helical, though other winding configurations including but not limited to changing the winding pitch (angle) of the filar(s) F relative to a longitudinal axis of the coil assembly 1, may also be used as the artisan will readily recognize. The winding configuration of the coils 91, 93, 95 may also be used to affect performance characteristics such as stiffness, flexibility, pushability, torquability and buckle resistance along the coils assembly 1.

In practice, the coils 91, 93 and 95 may be successively created by winding one or more filar(s) F around or about the axis AX. When inner liner L is present, the first inner coil 91 may be wound around liner L, followed by winding of the second middle coil 93 around the first inner coil 91 and, finally, winding the third outer coil 95 around the second middle coil 93. Alternatively, a removable cylindrical mandrel may be used to provide a form for the inner liner L and around which the coils 91, 93 and 95 may be successively formed by winding wires or filars F around the removable mandrel and defining axis AX. Following assembly of the coil assembly 1, the mandrel may be removed and an inner liner L, or a polymeric coating, may be inserted or applied to an inner lumen defined by the first coil 91.

Exemplary embodiments of a coil assembly 1 comprising first, second and third coils 91, 93 and 95 is illustrated in FIG. 5. Each of the first, second and third coils 91, 93, 95 further comprise a plurality of filar groups 97, wherein each filar group 97 comprises an exemplary number of 18 filars F that do not comprise a spacing between adjacent wires within the filar group 97. It is noted that the coil assembly 1 comprising first, second and third coils 91, 93 95 may be elastically deformed by stretching or bending the coil assembly 1 during vascular traversal or during an interventional procedure. The skilled artisan will recognize that gaps G between adjacent wires that are not attached or connected with each other may be created during a stretching or bending deformation. However, in an undeformed configuration, the wires or windings within a filar group 97 do not comprise a gap between adjacent wires.

The number of filar(s) F comprising a filar group 97 and/or the width or diameter of individual filar(s) F in first, second and third coils 91, 93, 95 may be constant or equal along the length of the coils 91, 93, 95, or may decrease in a distal direction along the coil(s) 91, 93, 95.

Moreover, one or more of coils 91, 93, and/or 95 may comprise one or more filar groups 97 defined by gaps G. In some embodiments, one or more of coils 91, 93, and/or 95 may not have a gap G defining filar groups 97, while the remaining coils may comprise one or more gaps G defining one or more filar groups 97.

Preferably, adjacent wires or filars F within a filar group 97 are not connected or attached to each other. As noted above, when the microcatheter 100 comprising coil assembly 1 bends to navigate a turn within the vasculature, the filar F elements may spread apart on the outer radius of the turn, and consequently the outer radius of the coil assembly 1, to accommodate the turn and to allow for sufficient flexibility to make the required turn. Hence, it may be preferable to not connect at least some of the adjacent filar(s) F to provide maximum flexibility.

However, in some embodiments, one or more adjacent filar(s) F within one or more filar groups 97 may be connected or attached to each other. In some embodiments, a proximal region of one or more of the coils 91, 93, 95 may comprise at least some adjacent filar(s) F that may be connected with each other while a distal region of the one or more coils 91, 93, 95 may comprise adjacent filar(s) F that are not connected with each other to increase flexibility of the distal region of the coil assembly 1.

Whether to connect or attach at least some adjacent filar(s) F within one or more of the coils 91, 93, 95 may be used to affect performance characteristics such as, inter alia, stiffness, flexibility, torquability, pushability and buckle resistance. In addition, the attachment or non-attachment of at least some adjacent filar(s) F of coils 91, 93, 95 may be used in combination with performance affecting features discussed herein.

As illustrated in FIG. 5, 18 filar(s) F within a filar group 97 is perhaps preferred but is also exemplary; other numbers of wires or filars F may be used. The number of filar(s) F within a filar group 97 is preferably between about 2 and about 50 filar(s) F, more preferably between about 6 and about 24 filar(s) F, more preferably between about 10 and 20 wires or filars F, and more preferably between about 16 and 18 filar(s) F. The stiffness, flexibility, pushability, torquability and/or buckle resistance may be affected by the selection of numbers of filar(s) F within a filar group 97. Accordingly, certain embodiments of coil assembly 1 may comprise one or more coils 91, 93, 95 comprising an equal number of filar(s) F within each filar group 97. Other embodiments may comprise a non-equal number of filar(s) F within each filar group 97. For example, and without limitation, a proximal region of one or more coils 91, 93, 95 may comprise one or more filar groups 97 that have a larger number of filar(s) F than the number of wires or filars F in one or more filar groups 97 in a distal region of the one or more coils 91, 93, 95 to achieve a stiffer proximal region and a more flexible distal region. The effective result of an unequal number of filar(s) F in filar groups 97 results in unequal spacing between adjacent filar groups 97 that have an unequal number of filar(s) F. A similar result is provided when filar(s) F of different widths are used within adjacent filar groups 97.

Thus, the number of filar(s) F in each filar group 97 within the coil assembly 1 comprising one or more of coils 91, 93, 95 may be used to adjust performance characteristics such as stiffness, flexibility, pushability, torquability and/or buckle resistance. Moreover, the number of filar(s) F in each filar group 97 within the coil assembly 1 comprising coils 91, 93, 95 may be used in combination with one or more of the performance affecting features discussed herein.

As illustrated in FIG. 5, one or more of the coils 91, 93, 95 may comprise at least one gap G between one or more adjacent pairs of filar groups 97 in some embodiments to achieve desirable balance of stiffness, flexibility, pushability, torquability and buckle resistance. If more than one gap G is provided on any coils, the gaps G may be longitudinally spaced apart from each other. The number gaps G over a defined distance (frequency of gaps G) may increase from a proximal portion of the catheter toward the distal portion. In addition or alternatively, in other embodiments, the width of the gaps G may increase in the distal direction along the length of the catheter 100. Alternatively, the width of the gaps G may decrease in the distal direction.

To provide a gap G, during construction of the outer coil 95, a 19 element filar may have one element removed to leave 18 filar elements and the gap G. Alternatively, one or more wires or filars may be wound about axis AX as discussed further herein.

At least one gap G may also be optionally provided in the first and second coils 91, 93. The width of the gap G may be preferably the width of a filar F or approximately 0.01 inches. In other embodiments, gap(s) G may be less than about 0.01 inches or greater than about 0.01 inches. The width of gap(s) G may be equal along the length of a coil assembly or may be non-equal. In some embodiments, the width of gaps along a proximal region of one or more of coil assemblies 91, 93, 95 may be of a width that is smaller or less than the width of gaps along a distal region of one or more of coil assemblies 91, 93, 95. The widths of gaps G may, in some embodiments, slowly increase moving from proximal to distal along one or more of coil assemblies 91, 93, 95. In other embodiments, a stepped change in gap G widths may occur in one or more coil assemblies 91, 93, 95 moving from proximal to distal.

In addition, gaps G may be used to define filar groups 97, wherein a gap G defines a space or separation between adjacent filar groups 97. In some embodiments, the gap G may define a circumferential space. In other embodiments a semi-circumferential space may be defined by gap G wherein one or more filar(s) F traverse a portion of the gap G between adjacent filar groups 97. In some embodiments a combination of circumferential gaps G and semi-circumferential gaps G may be provided.

Gaps G are preferred, but may not be present in some embodiments and may be present only along discrete regions of the catheter assembly 1 including only along discrete regions of one or more coils 91, 93, 95. When present, the gaps G may be used in combination with one or more of the performance affecting features discussed herein.

In one embodiment, all three of the first, second and third coils 91, 93, 95 may comprise a plurality of longitudinally spaced-apart gaps G. In addition to enhancing the flexibility of the microcatheter 1 while still providing the required pushability and torquability, the gaps G may be used to allow the flow of polymer around the during the assembly/construction process to effectively connect the coils 91, 93, 95 and the outer surface of the liner L. In other embodiments, one or more of coils 91, 93, 95 may comprise gaps G. In some embodiments, none of the coils 91, 93, 95 comprise gaps G.

Accordingly, gaps G may comprise a space defining not only a width as discussed above, but also a depth. If, for example, the outer coil 95 comprises a gap G, but the middle coil 93 does not also comprise a gap G that overlaps at least in part with the outer coil 95 gap G, then the depth of the outer coil 95 gap G will be effectively the size/height of the filar(s) F comprising outer coil 95. Generally, this single-coil gap G depth may be about 0.001 inches, or greater or less than about 0.001 inches, depending on the filar(s) F size or height of each coil.

In some embodiments, at least two of the coils 91, 93, 95 may comprise gaps G that overlap at some location(s) along the coil assembly 1. For example, at least one gap G of outer coil 95 may overlap with at least one gap of middle coil 93. Alternatively, at least one gap G of middle coil 93 may overlap with at least one gap G of inner coil 91. Thus, in this embodiment, a two-coil gap G depth may be provided along at least a portion of each of the overlapping gaps G. Generally, this two-coil overlapping gap G depth may be about 0.002 inches, or greater or less than about 0.002 inches, depending on the filar(s) F size or height. There may be portions of two gaps G that overlap and portions of the same two gaps that do not overlap. In this case, the overlapping depth may be about 0.002 inches, or greater or less than about 0.002 inches, and the non-overlapping gap depths for each coil may be about 0.001 inches, or greater or less than about 0.001 inches, depending on the filar(s) F size or height for each coil.

The first, second and third coils 91, 93 and 95 may be swaged to, among other things, to change the cross-sectional geometries of the wire assembly components and the space of the gaps. In some instances, swaging may control, block, reduce or eliminate fluid passages between adjacent windings of filar(s) F. It may also tend to feature flow of resins through the gaps G during the construction process. Swaged wires may also present a lower profile for passage through the patient's vessels which may accordingly reduce the depth of gaps G discussed herein.

In some embodiments, all three of the coils 91, 93, 95 may comprise gaps G that overlap at some location(s) along the coil assembly 1. For example, at least one gap G of outer coil 95 may overlap with at least one gap of middle coil 93 and those gaps G may overlap with at least one gap G of inner coil 91. Thus, in this embodiment, a three-coil gap G depth may be provided along at least a portion of each of the overlapping gaps G. Generally, if an exemplary filar(s) comprises a thickness of height of about 0.01 inches such that the resulting coil 91, 93 and/or 95 also comprise a depth of or thickness of about 0.01 inches, then this three-coil overlapping gap G depth may be about 0.03 inches, or greater or less than about 0.03 inches, depending on the filar(s) F size or height. There may be portions of three gaps G that overlap and portions of the same three gaps that do not overlap, or that overlap at only two coils. In this case, the three-coil overlapping depth may be about 0.03 inches, or greater or less than about 0.03 inches, a two-coil overlapping depth may be about 0.02 inches, or greater or less than about 0.02 inches, and the non-overlapping gap depths for each coil may be about 0.01 inches, or greater or less than about 0.01 inches, depending on the filar(s) F size or height for each coil.

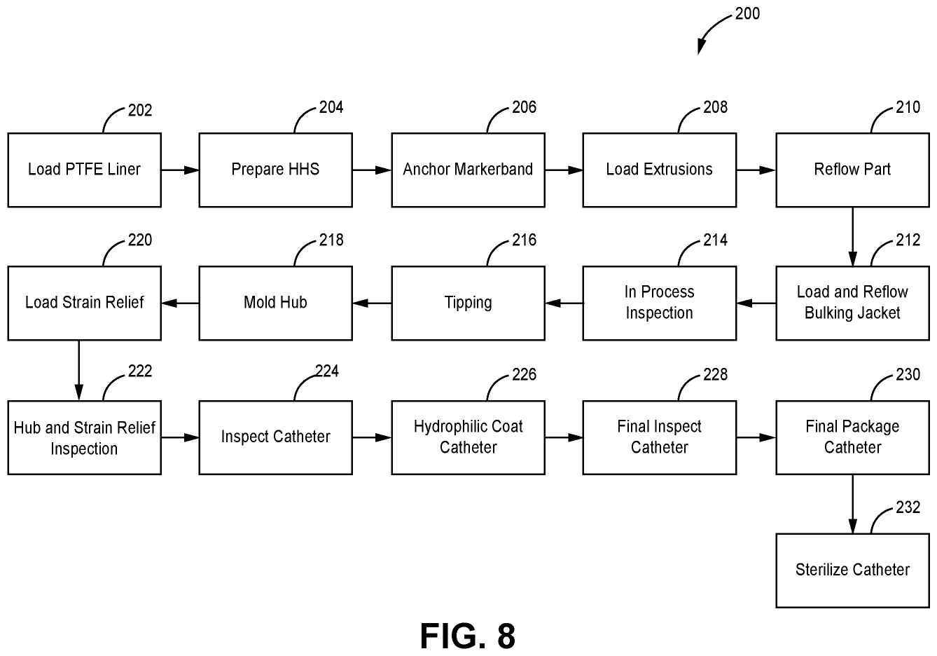

If a construction process is used that utilizes heat (e.g. heat shrinking or reflow) or the flow of polymeric materials (e.g. compression extrusion), then the gap G can allow flow of a polymeric material from the exterior toward the interior of the catheter (e.g. to the exterior of the liner L). If gaps G are provided in adjacent coils 91 or 93 or 95, then the gaps may be longitudinally staggered or alternatively, arranged to at least partially overlap and provide a pathway for resin flow during a construction process. See FIG. 8 for an exemplary manufacturing process.

Each filar(s) F may comprise an equal width, or the filar(s) F may comprise unequal widths. A preferred width is about 0.01 inches, though the filar(s) F may be less than or greater than 0.01 inches. The filar(s) F may comprise the same material throughout a coil 91, 93, and/or 95 and/or the coil assembly 1. Alternatively, more than one material may comprise filar(s) F for a coil 91, 93, 95. Still more alternatively, at least part of at least one of the coils 91, 93, and/or 95 may comprise filar(s) F that comprise a material at a proximal region that is different than a material at a distal region of the wires or filars F. As discussed briefly above, the number of filar(s) F and or the width or radius of individual filars F in one or more of coils 91, 93, 95 may be constant or equal along the length of one or more of the coils 91, 93, 95, or may decrease in a distal direction along one or more of the coil(s) 91, 93, 95.

The filar(s) F forming one or more of the coils 91, 93 and 95 may be swaged to add work hardening to the filars F and to change the cross sectional geometries of the wire assembly components and the space of the gaps. In some instances, swaging may control, block, reduce or eliminate fluid passages between filar winds. It may also tend to feature flow of resins through the gaps G during the construction process. Swaged filars F may also present a lower profile for passage through the patient's vessels and may be used to modify stiffness and/or flexibility characteristics, among other things.

Using these variables, the stiffness, flexibility, pushability and torquability may be optimized. For example, and without limitation, providing filar(s) F that are less than about 0.001 inches forming one or more coils 91, 93 or 95 may provide a more flexible coil assembly 1. Alternatively, a proximal region of one or more coils 91, 93, 95 may comprise filar(s) F that are wider than the width of filar(s) F at a distal region of one or more of the coil(s) 91, 93, 95.

Further, materials that are stiffer or more flexible may be used in a similar manner to provide filar(s) F that are wound to provide at least one coil 91, 93, 95 with a stiffer or more flexible material than the materials comprising the remaining coils 91, 93, 95. Alternatively, at least one part of at least one coil 91, 93, 95 may comprise a stiffer material that transitions along the length of the at least one coil to a less stiff, more flexible material. For example, a stiffer material may be used for filar(s) F in a proximal region of at least one of the coils 91, 93, 95 and a more flexible material may be used for filar(s) F in a distal region of the coils 91, 93, 95 to provide a more flexible coil assembly 1 at the distal region. Materials selection including filar(s) F width and/or filar(s) F material may be used alone or in combination to achieve the desired balance between stiffness, flexibility, pushability, torquability and buckle resistance.

In another embodiment, the flexibility of the filar(s) F may increase in the distal direction along inner liner L. In another embodiment, the stiffness of the filar(s) F may decrease in the distal direction, or sections of stiffer wires or filars F may be interposed between more flexible filar(s) F. The flexibility or stiffness may change gradually or it may change suddenly in different embodiments of the invention.

The filar(s) F forming one or more of the first, second and third coils 91, 93 and 95 may have a round or flattened (e.g. rectangular) cross sectional shape. Preferably, the filar(s) F are constructed from stainless steel; but alternative materials such as nitinol, gold, aluminum, silver and combinations thereof may be used. Examples of suitable materials include 316, 303, 302, 17-4PH, 17-7PH, 18-8 and 304V stainless steels and/or combinations thereof. In some instances, all of the filar(s) F of one or more of coils 91, 93, 95 may be identical, in other instances, different materials may be used for the coil(s) 91, 93 and 95, e.g., a coil may comprise filar(s) F constructed from different materials. In one embodiment all of the materials of all of the filar(s) F are the same material, e.g. a stainless steel. The individual filar(s) F may initially have a round cross section, but during the manufacturing process the wires or filars F may become flattened to provide a rectangular like cross-sectional shape during a construction step such as a step that includes swaging components together.

Preferably, the first, second and third coils 91, 93 and 95 are multi-filar coils. In one embodiment, one or more of the first, second and third coil assemblies 91, 93 and 95 are single filament wire coils comprising a single wire or filar F continuously wound as described above. One or more of the coils 91, 93 and 95 may be swaged to add work hardening to the wires and to change the cross sectional geometries of the wire assembly components and the space of the gaps. In some instances, swaging may control, block, reduce or climinate fluid passages between filar winds. It may also tend to feature flow of resins through the gaps G during the construction process. Swaged wires may also present a lower profile, i.e., crossing profile (outer diameter) for the microcatheter 1 which improves passage through the patient's vessels.

The coil assembly 1, and microcatheter 100, is preferably braid free to provide responsive torque characteristics and to provide axial strength. Surprisingly, it was found that a braid-free construction can provide a microcatheter with desirable properties such as torque response, pushability and flexibility, while retaining overall resistance to buckling. The absence of a braid was found to provide suitable mechanical characteristics while retaining a sufficient resistance to elongation. As noted however, some embodiments of the microcatheter disclosed herein may comprise a braid disposed along at least a portion of the inner liner L and/or disposed over one or more of the coils 91, 93, 95 and/or disposed between at least a portion of the lengths between two or more of the coils 91, 93 and/or 95. In some embodiments, the braid may extend to the distal end of the distal tip T. In other embodiments, the braid, when present, may terminate at a location that is proximal to the distal end of the distal tip T. In some embodiments, the braid may comprise a distal end that terminates at a point that is either proximal to, distal to, or at the same location as, the distal end of the coils 91, 93, and/or 95.

More generally, the outer diameter of catheter body 110 may gradually decrease moving from proximal to distal. The taper may be gradual or it may include a more discrete change or step in the outer diameter moving longitudinally. For example, and without limitation, the outer diameter may be closer to about 0.95 mm near the proximal portion P and closer to about 0.71 mm near the narrowing of the outer surface of the tip portion T.

FIG. 10 illustrates a partial cutaway view of an exemplary microcatheter 100 showing an exemplary three coil structure with outermost coil 95 and an optional marker band distal to the distal end of the three-coil structure. The marker band is illustrated as proximal to the distal end of the polymeric liner L, but may occupy other locations as well, including but not limited to extending to the distal end of the liner L. In addition, a marker material may be incorporated into the inner liner L and/or one or more of the coils 91, 93, 95. The optional marker band of FIG. 10, or incorporated marker material, may comprise materials that enhance visibility under a scan such as an Intravascular Ultrasound (IVUS), Optical Coherence Tomography (OCT), or other suitable imaging process.

Generally, the inner liner L may preferably extend to the distal end of the distal tip T. However, in other embodiments, the inner liner L may comprise a distal end that is proximal to the distal end of the distal tip T.

FIG. 8 provides an exemplary manufacturing process flow 200 for various embodiments of the disclosure. In operation 202, the polymeric liner is prepared for loading into the coil assembly. In some embodiments, the coil assembly will come pre-manufactured in a three-coil structure as described above. In some of these cases, an exemplary set of three coils may be of substantially equal length and wherein the distal ends of the coils 91, 93 and 95 are located at a common location.

In one embodiment, the polymeric inner liner may be inserted into the coil assembly. In another embodiment, the first coil may be wound around the polymeric inner liner, with subsequent coil(s) of the coil assembly wound around the first coil and, when a third coil is present, it may be would around the second coil.

In operation 206, a marker band or other material may be provided near a distal end of the polymeric liner. In step 208 extrusions are loaded and in step 210 a reflow of polymer process is conducted. As discussed herein, the reflow may have a path through the coil assembly through, e.g., gaps, in order to provide a seal with or against an outer surface of the inner polymeric liner.

In operation 212, a reflowed jacket is provided around the outer coil of the coil assembly. Operation 214 is a process inspection to ensure that the structure is thus far acceptable. Operation 216 includes the forming and attachment of the distal tip structure to the inner liner and coil assembly. Operation 218 molds the proximal hub structure and operation 220 provides for forming of the strain relief structure. Operation 222 is an inspection of the formed hub and strain relief structures and operation 224 is an overall inspection of the catheter.

Operation 226 is a coating of the catheter with a hydrophilic material and operation 228 is a final catheter inspection. In operation 230, the finished microcatheter is packaged and in operation 232 the packaged microcatheter is sterilized.

Generally, the inventors have discovered that, for microcatheters used for collateral vessel access generally, and for microcatheters commonly used for, e.g, retrograde or antegrade access to a lesion or site of interest, the following functional elements provide an enhanced crossability of vasculature and lesions:

A bi-directional rotational capability, wherein the microcatheter is configured to be rotated in at least a first clockwise direction and at least a first counterclockwise direction, and wherein substantially similar torqueing forces are produced at a distal end of the microcatheter by the clockwise and counterclockwise rotations. The following torqueing force data and ranges have been found to provide improved bi-directional rotational capability and the resulting crossability functionality.

For example, the exemplary microcatheters of the present disclosure preferably provide a torqueing force produced by a first clockwise rotation of one revolution of the microcatheter and a torqueing force produced by a first counterclockwise rotation of one revolution of the microcatheter are each within the range of about 0.08 to about 0.1 ounce force-inch.

Further, the exemplary microcatheters of the present disclosure preferably provide torqueing forces produced by a first clockwise rotation and a first counterclockwise rotation that are within about 0.02 ounce force-inch of each other.

In addition, the exemplary microcatheters of the present disclosure preferably provide torqueing forces produced by a second clockwise rotation and a second counterclockwise rotation that are within about 0.02 ounce force-inch of each other.

The exemplary microcatheters of the present disclosure also preferably provide torqueing forces produced by a third clockwise rotation and a third counterclockwise rotation that are within about 0.02 ounce force-inch of each other.

The exemplary microcatheters of the present disclosure preferably provide torqueing forces produced by a fourth clockwise rotation and a fourth counterclockwise rotation that are within about 0.02 ounce force-inch of each other.

The exemplary microcatheters of the present disclosure also preferably provide torqueing forces produced by a fifth clockwise rotation and a fifth counterclockwise rotation that are within about 0.02 ounce force-inch of each other.

The exemplary microcatheters of the disclosure further provide a difference in magnitude of torqueing force produced by a first clockwise rotation of one revolution and a second clockwise rotation of one revolution that is within the range of about 0.08 to about 0.1 ounce force-inch, wherein a difference in magnitude of torqueing forces produced by a first counterclockwise rotation of one revolution of the microcatheter and a second counterclockwise rotation of one revolution of the microcatheter is within the range of about 0.08 to about 0.1 ounce force-inch.

In addition, the exemplary microcatheters of the disclosure provide a difference in magnitude of torqueing forces produced by the second clockwise rotation and by a third clockwise rotation of one revolution of the microcatheter that is within the range of about 0.08 to about 0.1 ounce force-inch, wherein a difference in magnitude of torqueing forces produced by the second counterclockwise rotation and by a third counterclockwise rotation of one revolution of the microcatheter is within the range of about 0.08 to about 0.1 ounce force-inch.

Further, the exemplary microcatheters of the disclosure provide a difference in magnitude of torqueing forces produced by the third clockwise rotation and by a fourth clockwise rotation of one revolution of the microcatheter that is within the range of about 0.08 to about 0.1 ounce force-inch, wherein a difference in magnitude of torqueing forces produced by the third counterclockwise rotation and by a fourth counterclockwise rotations of one revolution of the microcatheter is within the range of about 0.08 to about 0.1 ounce force-inch.

The exemplary microcatheters of the disclosure further provide a difference in magnitude of torqueing forces produced by the fourth clockwise rotation and by a fifth clockwise rotation of one revolution of the microcatheter is within the range of about 0.08 to about 0.1 ounce force-inch, wherein a difference in magnitude of torqueing forces produced by the fourth counterclockwise rotation and by a fifth counterclockwise rotations of one revolution of the microcatheter is within the range of about 0.08 to about 0.1 ounce force-inch.

In addition, when the distal end of the microcatheters of the disclosure comprise a flexibility that, when within preferred ranges, contributes to improved crossability characteristics.

For example, when an applied force causes the distal end of the microcatheters of the present invention to deflect or bend away from a longitudinal axis a distance of 0-2 mm, a preferred range for the slope of the applied force is within the range of about (y=0.085x+b) to about (y=0.13x+b).

When an applied force causes the distal end of the microcatheters of the present invention to deflect or bend away from a longitudinal axis a distance of 0-4 mm, a preferred range for the slope of the applied force is within the range of about (y=0.07x+b) to about (y=0.12x+b).

When an applied force causes the distal end of the microcatheters of the present invention to deflect or bend away from a longitudinal axis a distance of 0-6 mm, a preferred range for the slope of the applied force is within the range of about (y=0.08x+b) to about (y=0.1x+b).

When an applied force causes the distal end of the microcatheters of the present invention to deflect or bend away from a longitudinal axis a distance of 0-8 mm, a preferred range for the slope of the applied force is within the range of about (y=0.07x+b) to about (y=0.09x+b).

Similarly, a preferred force range that provides for a deflection or a bending of the distal end of the microcatheters of the present disclosure from 0 mm to 2 mm away from a longitudinal axis is about 0.09 to about 0.130 g/mm.

A preferred force range that provides for a deflection or a bending of the distal end of the microcatheters of the present disclosure from 0 mm to 4 mm away from a longitudinal axis is about 0.085 to about 0.12 g/mm.

A preferred force range that provides for a deflection or a bending of the distal end of the microcatheters of the present disclosure from 0 mm to 6 mm away from a longitudinal axis is about 0.085 to about 0.1 g/mm.

A preferred force range that provides for a deflection or a bending of the distal end of the microcatheters of the present disclosure from 0 mm to 8 mm away from a longitudinal axis is about 0.07 to about 0.09 g/mm.

Table 1 below provides two working and non-limiting examples of microcatheters according to the present disclosure. As noted above, microcatheters may be used generally to obtain collateral vessel access as well as other types of vessel access. In some cases microcatheters commonly used for retrograde procedures may present the best option to a physician, while a physician may prefer microcatheters commonly referred used for antegrade procedures in other cases. Table 1 provides two exemplary microcatheters of the disclosure that may be used in a retrograde procedure or in an antegrade procedure.

TABLE 1
Microcatheter Working Examples.

| | | | | Exam- | Exam- |
|---|---|---|---|---|---|
| | | | | ple 1 | ple 2 |
| | Ex. of Material; | Range (cm) | | (cm) | (cm) |
| Ref. | Description | unless | | unless | unless |
| # | (cm) unless noted | noted | | noted | noted |
| 1 | Coil assembly; braid | | | | |
| | free tube; | | | | |
| | Metals such as nitinol or | | | | |
| | stainless steel or an alloy | | | | |
| | thereof; or a Cu-ZN-X | | | | |
| | allow where X = Fe, | | | | |
| | Al or the like | | | | |
| 2 | Polymer Liner | | | | |
| | PTFE; polyamides | | | | |
| 3 | Distal Tip and | 0.1-5 | | | |
| | outer layer; | | | | |
| | 75A-95A Carbothane | | | | |
| 4 | 75A-95A Carbothane | 1-7 | | 5 | 5 |
| | outer layer | | | | |
| 5 | 25D-60D Polymer or | 3-12 | | 7 | 7 |
| | copolymer with possible | | | | |
| | elastomer added such as | | | | |
| | polymeric elastomer | | | | |
| | such as Hytrel or | | | | |
| | PEBAX .002-0.004 | | | | |
| | inch Wall Thickness | | | | |
| 6 | 35D-60D Polymer or | 3-14 | | 8 | 8 |
| | copolymer with possible | | | | |
| | elastomer added such as | | | | |
| | polymeric elastomer | | | | |
| | such as Hytrel or | | | | |
| | PEBAX .002-0.004 | | | | |
| | inch Wall Thickness | | | | |
| 7 | 45D-65D Polymer or | 10-20 | | 15 | 15 |
| | copolymer with possible | | | | |
| | elastomer added such as | | | | |
| | polymeric elastomer | | | | |
| | such as Hytrel or | | | | |
| | PEBAX .0025- | | | | |
| | 0.0045 inch Wall | | | | |
| | Thickness | | | | |
| 8 | 58D-68D Polymer or | 10-20 | | 15 | 15 |
| | copolymer with possible | | | | |
| | elastomer added such as | | | | |
| | polymeric | | | | |
| | elastomer such as | | | | |
| | Hytrel or Vestamid | | | | |
| | or PEBAX | | | | |
| | .0025-0.0045 inch Wall | | | | |
| | Thickness | | | | |
| 9 | 67D-77D Polymer or | 25-35 | | 30 | 30 |
| | copolymer with possible | | | | |
| | elastomer added | | | | |
| | such as 67D-77D | | | | |
| | polymeric | | | | |
| | elastomer such | | | | |
| | as Hytrel or | | | | |
| | PEBAX .003- | | | | |
| | 0.005 inch Wall | | | | |
| | Thickness | | | | |
| 10 | A polymer such | 50-70 | | 60 | 60 |
| | as Grilamid, Pellethane, | | | | |
| | AESNO Nylon 12 | | | | |
| | 0.003- 0.005 inch Wall | | | | |
| | thickness | | | | |
| 12 | A polymer such | 30-70 | | 51 | 51 |
| | as Grilamid, Pellethane, | | | | |
| | AESNO Nylon 12 | | | | |
| | (Bulking Jacket) | | | | |
| 13 | Hub; polymer | | | | |
| 14 | Length | 40-55 | | 32-46.9 | 46.9 |
| 15 | Length | 60-85 | | 58.1-73.1 | 73.1 |
| 16 | Length | 25-35 | | 29.9 | 29.9 |
| 17 | Length | 3-6 | | 4.61 | 4.61 |
| 18 | Length | 3-8 | | 5.5 | 5.5 |
| 19 | Length | 0.5-3 | mm | 1.5 mm | 1.5 mm |
| | | | | +/− | +/− |
| | | | | 0.5 mm | 0.5 mm |
| 20 | Max Length or Dia | 0.01-0.025 | inches | 0.019 | 0.019 |
| | | | | (inches) | (inches) |
| 22 | Working Length | About 115 cm | | 135 cm | 150 cm |
| | | and | | +/− | +/− |
| | | about 200 cm | | 2 cm | 2 cm |
| 23 | Length | 45-75 | cm | 45 | 60 cm |
| | Distance from the distal | 0-5 | mm | 1 mm | 1 mm |
| | ends of the | | | | |
| | coil assembly 1 | | | | |
| | to the distal end of distal | | | | |
| | tip. | | | | |

### WORKING EXAMPLES AND COMPETITIVE PRODUCTS

Working Example 1 is a bi-directional torque test that measures torqueing forces for clockwise and counterclockwise rotation(s) of the tested microcatheters.

Working Example 2 is a deflection, or flexibility, test that measures the amount of force applied to cause the distal end of each tested microcatheter to deflect or bend away a selected distance from a longitudinal axis through the microcatheter.

### The Tested Microcatheters

A summary table of the relevant characteristics of the tested microcatheters is provided in Table 2. The competitive devices A and B are currently marketed microcatheters that are commonly used in retrograde procedures. The comparison microcatheter that is an exemplary embodiment of the disclosure may be used in a retrograde procedure, but is specifically not limited to a retrograde procedure and, therefore, may also be used in an antegrade procedure.

TABLE 2

| | Exemplary | | |
|---|---|---|---|
| | Embodiment | | |
| | of the | | |
| | Disclosure | Competitor A | COMPETITOR B |
| Polymer | Polymer liner | Polymer liner | Polymer liner extends |
| liner | extending | stopping appx. 6 | to the distal tip of the |
| | all the way | mm from the | microcatheter. |
| | to the distal | distal end of the | |
| | end of the | microcatheter. | |
| | microcatheter. | | |
| Braid | No Braid | Inner Braid, stops | Inner Braid |
| | | appx. 145 mm | surrounding the |
| | | from the distal end | polymer liner, braid |
| | | of the | stops 4 mm from |
| | | microcatheter. | distal end of the |
| | | | microcatheter. |
| Coil | Triple coil, | Single coil | Dual coil, each coil |
| Structure | each coil | surrounding the | having a different |
| | having a | Braid, stops appx | winding direction. |
| | winding | 6 mm from the | The coils terminate |
| | direction | distal end of the | proximal to the distal |
| | different from | microcatheter, i.e., | end of the |
| | the winding | the same distance | microcatheter and the |
| | direction(s) | from the distal end | distal end of the braid. |
| | of adjacent | as the polymer | Dual coil transitions to |
| | coil(s). | liner. | a single coil at a distal |
| | | | region of the |
| | | | microcatheter. |
| Distal | The distal | Braid distal end is | Dual coil transition to |
| End(s) of | end of the | located proximal | single coil occurs |
| Coils. | triple coil | to the distal end of | proximal to the distal |
| | is located | the single coil | end of the |
| | appx 1 mm | which, in turn, is | microcatheter and |
| | proximally | proximal to the | proximal of the distal |
| | from the | distal end of the | end of the braid. |
| | distal end of | microcatheter. | Distal end of the single |
| | the | | coil is proximal of the |
| | microcatheter. | | distal end of the braid |
| | | | which is proximal of |
| | | | the distal end of the |
| | | | microcatheter. |
| Gaps in | Gaps, defining | No gaps | No gaps |
| Coils | groups of | | |
| | 18 filars | | |
| | along the | | |
| | length of | | |
| | each of the | | |
| | coils. | | |

### Working Example 1-Bi-Directional Torque Testing and Competitive Comparisons

A bi-directional torque test was conducted on the tested microcatheters using a test platform and method that are further described below. The test compares torqueing forces produced by the microcatheters after one or more rotations in a clockwise and/or counterclockwise direction. An exemplary embodiment of the present disclosure was tested, wherein the tested exemplary embodiment's structure is within the descriptions of Table 1 and Table 2, together with the selected currently marketed and tested microcatheters as described in Table 2.

### Torquing Force Production Test Setup and Method

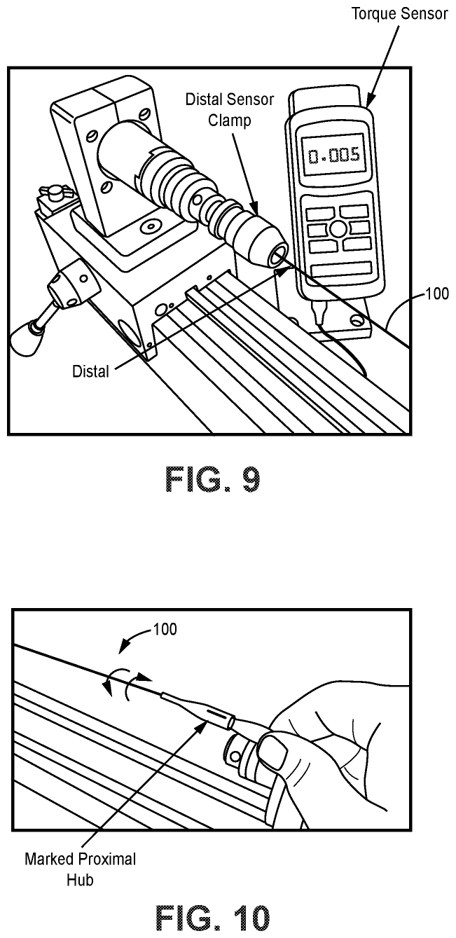

With reference to FIGS. 9 and 10, the distal tip of the test microcatheter is clamped within a torque sensor and a guide wire is inserted through the microcatheter hub and lumen through the hub and microcatheter shaft. The hub is marked to allow identification of rotational position, and to facilitate turning or rotating the microcatheter a predetermined amount, e.g. one rotation, in either the clockwise or counterclockwise direction. The test method used to generate torqueing force data follows:
1. Clamp the distal tip in a torque sensor with a short 0.014″ mandrel in the ID. See FIG. 8.
2. Insert a guidewire (0.014″) or simulated (0.014″) guidewire mandrel through the hub till it contacted the tip mandrel. See FIG. 9.
3. Mark the hub to indicate a rotational location of a zero-degree rotation point. See FIG. 10.
4. Rotate the hub one revolution or approximately 360 degrees, in either the clockwise or the counterclockwise direction. Ensure the mandrel moves freely, and read the torqueing force magnitude produced by the rotation from the torque sensor display.
5. Repeat a second, third, etc., rotation in the selected rotational direction, e.g., clockwise, until a failure occurs or a predetermined number of rotations are reached, and reading the torqueing force magnitude produced by each rotation from the torque sensor display.
6. Repeat the rotations in the other rotational direction, e.g., counterclockwise, and read the produced torqueing force magnitudes displayed by the torque sensor.

In generating the torque force test data shown below in Table 3, several samples, e.g., 3-5 samples, for each microcatheter were tested. The averages of the individual test runs are provided in Table 3.

### Table 3 : Torque Force Production Data Summary

Microcatheter Torque Force Magnitude Data Summary

| | Exemplary | Competitor A | Competitor B |
|---|---|---|---|
| | Embodiment | Torque Force | Torque Force |
| Rotations and | Torque Force | (ounce | (ounce |
| Direction* | (ounce force-in) | force-in) | force-in) |
| 1^{st} CW rotation | 0.101 | 0.067 | 0.225 |
| 2^{nd} CW rotation | 0.19 | 0.120 | 0.423 |
| 3^{rd} CW rotation | 0.28 | 0.157 | 0.613 |
| 4^{th} CW rotation | 0.368 | 0.192 | NA |
| 5^{th} CW rotation | 0.446 | NA | NA |
| 1^{st} CCW rotation | 0.091 | 0.027 | 0.017 |
| 2^{nd} CCW rotation | 0.177 | 0.070 | 0.029 |
| 3^{rd} CCW rotation | 0.272 | 0.110 | 0.335 |
| 4^{th} CCW rotation | 0.358 | 0.143 | NA |
| 5^{th} CCW rotation | 0.44 | 0.44 | NA |

*CW = clockwise rotational direction, and CCW = counterclockwise rotational direction.

### Working Example 2-Flexibility Testing of Distal Region and Competitor Comparisons

A flexibility test of a 25 cm distal region was conducted on the tested microcatheters using a test platform and method that are further described below. The test compares the forces required to deflect or bend an approximately 25 cm distal end region of the tested catheters a defined distance away from a longitudinal axis. An exemplary embodiment of the present disclosure was tested, together with the selected currently marketed microcatheters as described in Table 2.

### Flexibility of Distal Region Test Setup and Method

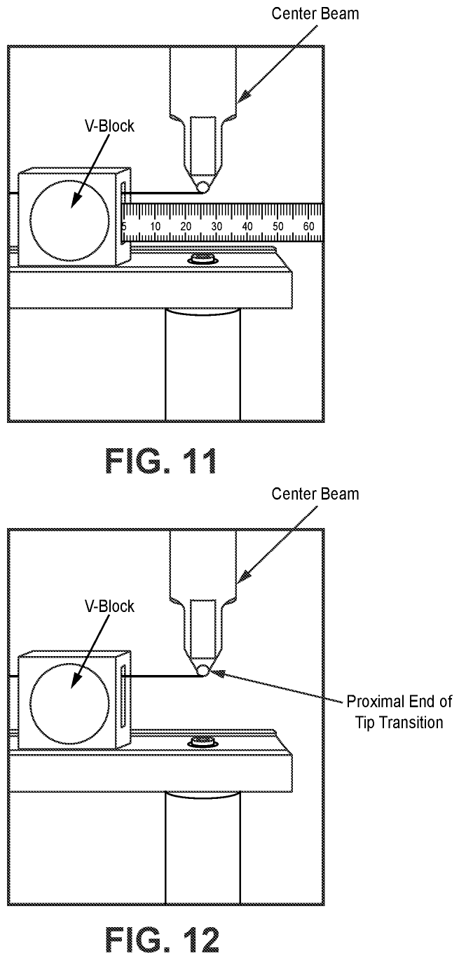

A flexibility test setup and method include a v-block and a center beam as shown in FIGS. 11 and 12. The flexibility of a distal end region, defined as about 25 cm from the distal end of the tested microcatheter, was tested by applying force to the distal end region at an approximately orthogonal direction to a longitudinal axis of the microcatheter. The applied force required to move, deflect or bend the distal tip a predetermined distance, e.g., about 2 mm, about 4 mm, about 6 mm and about 8 mm was recorded. The specific test method steps follow:
1. Position v-block approximately 25 mm from center beam.
2. Position catheter tip so that the proximal end of the tip transition is under the center beam.
3. Bring the center beam within one click of the positioning wheel of adding load reading.
4. Zero distance and Zero load reading
5. Run test, deflect the distal tip to about 2 mm, about 4 mm, about 6 mm, and about 8 mm.
6. Record the forces required to reach the deflection distances.

In generating the flexibility test data shown below in Table 4, several samples, e.g., 3-5 samples, for each microcatheter were tested. The averages of the individual test runs are provided in Table 4.

TABLE 4
Flexibility Data Summary
Microcatheter Distal End Region Deflection Testing Force Results

| | g/mm @ | g/mm @ | g/mm @ | g/mm @ |
|---|---|---|---|---|
| | app. 2 mm | app. 4 mm | app. 6 mm | app. 8 mm |
| Microcatheter | deflection | deflection | deflection | deflection |
| Exemplary | 0.122 g/mm | 0.109 g/mm | 0.099 g/mm | 0.088 g/mm |
| Embodiment | | | | |
| Competitor A | 0.082 g/mm | 0.077 g/mm | 0.074 g/mm | 0.068 g/mm |
| Competitor B | 0.135 g/mm | 0.125 g/mm | 0.114 g/mm | 0.100 g/mm |

In addition to the deflection force required to deflect or bend the distal tip of each tested microcatheter to the designated deflection distances, i.e., about 2 mm, about 4 mm, about 6 mm and about 8 mm, the slopes of the tested data were also calculated at the designated deflection distances as shown in Table 5 below. Each of the slopes in Table 5 are averages of several test runs. A line equation may be provided for each of the average slope values. For example, for the Exemplary Embodiment at 2 mm of deflection, the line equation for the slope is y=0.124x+b. The remaining slope values in Table 5 are also amenable to a line equation with variables for y and intercept (b).

TABLE 5
Flexibility of Distal End Region-Slope Results
Microcatheter Deflection Testing Slope of Testing Force Results

| | Slope | Slope | Slope | Slope |
|---|---|---|---|---|
| | @ 2 mm | @ 4 mm | @ 6 mm | @ 8 mm |
| Microcatheter | Deflection | Deflection | Deflection | Deflection |
| Exemplary | 0.124x | 0.077x | 0.097x | 0.087x |
| Embodiment | | | | |
| Competitor A | 0.079x | 0.109x | 0.073x | 0.068x |
| Competitor B | 0.136x | 0.126x | 0.116x | 0.102x |

A catheter 100 constructed using the above teachings and discoveries in various combinations can be provided with highly desirable combination of features contributing to, inter alia, improved crossability of vasculature and lesions, such as stiffness and axial force transmission; flexibility and torque response, peak tracking force, deflection force, kink resistance, and buckling resistance.

Exemplary embodiments of some of the disclosed microcatheters follow:

Embodiment 1: A microcatheter comprising:
a polymeric inner liner comprising a proximal end, a distal end and a length, and
defining a lumen comprising an inner diameter;
a coil assembly comprising
a first innermost coil comprising a proximal end, a distal end and a length, wherein the first innermost coil comprises one or more filars wound around the polymeric inner liner in a first wind direction,
a second middle coil, comprising a proximal end, a distal end and a length, wherein the second middle coil comprises one or more filars wound about the first, innermost coil in a second wind direction different than the first wind direction, and
a third outermost coil comprising a proximal end, a distal end and a length, wherein the third outermost coil comprises one or more filars wound about the second middle coil in a direction different than the second wind direction,
wherein the distal end of the third outermost coil is located proximal to the distal ends of the first innermost coil and the second middle coil, and
wherein the distal ends of the first innermost coil, the second middle coil and the third outermost coil each terminate at the same location;
a polymeric outer layer surrounding the coil assembly;
a distal tip formed of at least one polymer and comprising a proximal end and a distal end and surrounding the inner liner, the distal tip operatively connected with the polymeric outer layer, and wherein the inner liner extends to the distal end of the distal tip; and
a hub operatively connected with a proximal region of the coil assembly and defining a lumen and a proximal inlet to, and in fluid communication with, the lumen, the proximal inlet defined by the hub and the lumen defined by the hub each in fluid communication with the lumen defined by the inner liner.

Embodiment 2: The microcatheter of embodiment 1, wherein the distal tip further surrounds a proximal portion of the coil assembly.

Embodiment 3: The microcatheter of embodiment 1, wherein at least some of the filars of at least a portion of the coil assembly are swaged to reduce the width and/or height of the swaged filars.

Embodiment 4: The microcatheter of embodiment 1, wherein the microcatheter comprises an outer diameter that decreases in the distal direction.

Embodiment 5: The microcatheter of embodiment 1, wherein the microcatheter comprises proximal, transition and distal sections, wherein the distal section outer diameter is about 2.1 F.

Embodiment 6: The microcatheter of embodiment 1, wherein the length of the first innermost coil is longer than the length of the third outermost coil.

Embodiment 7: The microcatheter of embodiment 1, wherein the length of the second middle coil is longer than the length of the third outermost coil.

Embodiment 8: The microcatheter of embodiment 1, wherein at least one of the first innermost coil, second middle coil and third outermost coil are wound in a spiral configuration.

Embodiment 9: The microcatheter of embodiment 1, wherein the microcatheter is configured to be rotated bi-directionally and resist lengthening and shortening during rotation.

Embodiment 10: The microcatheter of embodiment 1, wherein the distal ends of the first innermost coil, the second middle coil and the third outermost coil are located less than 5 mm from the distal end of the distal tip.

Embodiment 11: The microcatheter of embodiment 1, wherein the distal ends of the first innermost coil, the second middle coil and the third outermost coil are less than 2 mm from the distal end of the distal tip.

Embodiment 12: The microcatheter of embodiment 1, wherein the distal ends of the first innermost coil, the second middle coil and the third outermost coil are located approximately 1 mm from the distal end of the distal tip.

Embodiment 13: The microcatheter of embodiment 1, wherein the distal ends of the first innermost coil, the second middle coil and the third outermost coil are located less than 1 mm from the distal end of the distal tip.

Embodiment 14: The microcatheter of embodiment 1, the polymer outer layer comprising a section adjacent to the distal tip comprising a shore hardness of 35 D or less and a length of approximately 15 cm.

Embodiment 15: The microcatheter of embodiment 1, wherein the distal tip comprises a distal region of decreasing taper in the distal direction.

Embodiment 16: The microcatheter of embodiment 1, wherein the distal tip comprises a distal region of decreasing outer diameter in the distal direction.

Embodiment 17: The microcatheter of embodiment 1, further comprising the polymer outer layer decreasing in shore hardness in the distal direction.

Embodiment 18: The microcatheter of embodiment 1, wherein at least one of the first innermost coil, the second middle coil and the third outermost coil comprises at least two groups of two or more windings of the one or more filars,
wherein there is no gap between adjacent windings within the at least two groups of two or more windings of the one or more filars, and
wherein there is a gap between adjacent groups of windings.

Embodiment 19: The microcatheter of claim embodiment 18, wherein each group of windings comprises between about 6 and 24 windings.

Embodiment 20: The microcatheter of embodiment 18, wherein each group of windings comprises between about 10 and about 20 windings.

Embodiment 21: The microcatheter of embodiment 18, wherein each group of windings comprise 18 windings.

Embodiment 22: The microcatheter of embodiment 18, wherein each group of windings comprises an equal number of windings.

Embodiment 23: The microcatheter of embodiment 18, wherein the number of windings within each group of windings decreases in the distal direction along at least a portion of the length of at least one of the first innermost coil, the second middle coil and the third outermost coil.

Embodiment 24: The microcatheter of embodiment 18, wherein the one or more wires comprise a width and wherein the gap between adjacent groups is approximately equal to the width of the one of more wires.

Embodiment 25: The microcatheter of embodiment 18, wherein the gap between adjacent groups is greater than or equal to 0.01 inches.

Embodiment 26: The microcatheter of embodiment 18, wherein the gap between adjacent groups is less than 0.01 inches.

Embodiment 27: The microcatheter of embodiment 18, wherein a width of the gap between adjacent groups of windings increases in the distal direction along at least a portion of the length of at least one of the first innermost coil, the second middle coil and the third outermost coil.

Embodiment 28: The microcatheter of embodiment 1, wherein the first, second and third coils are constructed from the same material.

Embodiment 29: The microcatheter of embodiment 1, wherein the first, second and third coils are constructed from different materials.

Embodiment 30: The microcatheter of embodiment 1, wherein the microcatheter is an antegrade catheter or a retrograde catheter.

Embodiment 31: The microcatheter of claim 1, wherein the microcatheter does not comprise a braid.

Embodiment 32: The microcatheter of embodiment 1, comprising one or more of embodiments 2-31.

Further modifications and improvements may additionally be made to the device and method disclosed herein without departing from the scope of the present invention. Accordingly, it is not intended that the invention be limited, except as by the appended claims. Depiction of different features as combinations of materials and geometries is intended to highlight different functional aspects and does not necessarily imply that such features must be realized by the described materials and geometries for such components. Rather, functionality associated with one or more geometries and materials may be performed by separate or different geometries or materials.

1. A microcatheter comprising:
a polymeric inner liner comprising a proximal end, a distal end and a length, and defining a lumen comprising an inner diameter;
a coil assembly surrounding a portion of the length of the polymeric inner liner; and
wherein the microcatheter is configured to rotate in a clockwise and in a counterclockwise direction to produce torqueing forces at a distal end of the microcatheter, and
wherein a torqueing force produced by a first clockwise rotation of one revolution of the microcatheter and a torqueing force produced by a first counterclockwise rotation of one revolution of the microcatheter are each within the range of about 0.08 to about 0.1 ounce force-inch.

2. The microcatheter of claim 1, wherein the polymeric inner liner extends distally to a distal end of the microcatheter.

3. The microcatheter of claim 1, wherein the coil assembly comprises a first inner coil surrounding at least a portion of a length of the polymeric inner liner, a second middle coil surrounding at least a portion of a length of the first inner coil, and a third outermost coil surrounding at least a portion of a length of the second middle coil, wherein the coil assembly comprises a distal end that is located proximal to the distal end of the microcatheter.

4. The microcatheter of claim 1, wherein the torqueing forces produced by the first clockwise rotation and the first counterclockwise rotation are within about 0.02 ounce force-inch of each other.

5. The microcatheter of claim 1, wherein the torqueing forces produced by a second clockwise rotation of one revolution of the microcatheter and a second counterclockwise rotation of the microcatheter are within about 0.02 ounce force-inch of each other.

6. The microcatheter of claim 1, wherein the torqueing forces produced by a third clockwise rotation of one revolution of the microcatheter and a third counterclockwise rotation of one revolution of the microcatheter are within about 0.02 ounce force-inch of each other.

7. The microcatheter of claim 1, wherein the torqueing forces produced by a fourth clockwise rotation of one revolution of the microcatheter and a fourth counterclockwise rotation of one revolution of the microcatheter are within about 0.02 ounce force-inch of each other.

8. The microcatheter of claim 1, wherein the torqueing forces produced by a fifth clockwise rotation of one revolution of the microcatheter and a fifth counterclockwise rotation of one revolution of the microcatheter are within about 0.02 ounce force-inch of each other.

9. The microcatheter of claim 1, wherein the microcatheter is configured to be used in perform a retrograde access of an intravascular site of interest.

10. The microcatheter of claim 1, wherein the microcatheter is configured to be used to perform an antegrade access of an intravascular site of interest.

11. A microcatheter comprising:
a polymeric inner liner comprising a proximal end, a distal end and a length, and defining a lumen comprising an inner diameter;
a coil assembly surrounding a portion of the length of the polymeric inner liner,
wherein the microcatheter is configured to rotate in a clockwise and in a counterclockwise direction to produce a torqueing force at a distal end of the microcatheter, and
wherein a difference in magnitude of torqueing forces produced by a first clockwise rotation of one revolution of the microcatheter and by a second clockwise rotation of one revolution of the microcatheter is within the range of about 0.08 to about 0.1 ounce force-inch, and
wherein a difference in magnitude of torqueing forces produced by a first counterclockwise rotation of one revolution of the microcatheter and by a second counterclockwise rotation of one revolution of the microcatheter is within the range of about 0.08 to about 0.1 ounce force-inch.

12. The microcatheter of claim 11, wherein the polymeric inner liner extends distally to a distal end of the microcatheter.

13. The microcatheter of claim 11, wherein the coil assembly comprises a first inner coil surrounding at least a portion of a length of the polymeric inner liner, a second middle coil surrounding at least a portion of a length of the first inner coil, and a third outermost coil surrounding at least a portion of a length of the second middle coil, wherein the coil assembly comprises a distal end that is located proximal to the distal end of the microcatheter.

14. The microcatheter of claim 11, wherein a difference in magnitude of torqueing forces produced by the second clockwise rotation and by a third clockwise rotation of one revolution of the microcatheter is within the range of about 0.08 to about 0.1 ounce force-inch, and
wherein a difference in magnitude of torqueing forces produced by the second counterclockwise rotation and a third counterclockwise rotations is within the range of about 0.08 to about 0.1 ounce force-inch.

15. The microcatheter of claim 11, wherein a difference in magnitude of torqueing forces produced by the third clockwise rotation and by a fourth clockwise rotation of one revolution of the microcatheter is within the range of about 0.08 to about 0.1 ounce force-inch, and
wherein a difference in magnitude of torqueing forces produced by the third counterclockwise rotation and by a fourth counterclockwise rotations is within the range of about 0.08 to about 0.1 ounce force-inch.

16. The microcatheter of claim 11, wherein a difference in magnitude of torqueing forces produced by the fourth clockwise rotation and by a fifth clockwise rotation of one revolution is within the range of about 0.08 to about 0.1 ounce force-inch, and
wherein a difference in magnitude of torqueing forces produced by the fourth counterclockwise rotation and by a fifth counterclockwise rotations of one revolution of the microcatheter is within the range of about 0.08 to about 0.1 ounce force-inch.

17. The microcatheter of claim 11, wherein the microcatheter is configured to be used in perform a retrograde access of an intravascular site of interest.

18. The microcatheter of claim 11, wherein the microcatheter is configured to be used to perform an antegrade access of an intravascular site of interest.

19. A microcatheter comprising:
a polymeric inner liner comprising a proximal end, a distal end and a length, and defining a lumen comprising an inner diameter;
a coil assembly surrounding a portion of the length of the polymeric inner liner, wherein the microcatheter is configured to rotate in a clockwise and in a counterclockwise direction to produce a torqueing force at a distal end of the microcatheter, and
wherein over a plurality of clockwise and counterclockwise rotations, each rotation comprising one revolution of the, a first clockwise rotation of the microcatheter produces a torqueing force that is within the range of about 0.08 to about 0.1 ounce force-inch,
wherein a first counterclockwise rotation of one revolution of the microcatheter produces a torqueing force that is within the range of about 0.08 to about 0.1 ounce force-inch,
wherein a torqueing force within the range of about 0.08 to about 0.1 ounce force-inch is produced for each subsequent one of the plurality of clockwise rotations, and
wherein a torqueing force within the range of about 0.08 to about 0.1 ounce force-inch is produced for each subsequent one of the plurality of counterclockwise rotations.

20. The microcatheter of claim 19, wherein the polymeric inner liner extends distally to a distal end of the microcatheter and wherein the coil assembly comprises a first inner coil surrounding at least a portion of a length of the polymeric inner liner, a second middle coil surrounding at least a portion of a length of the first inner coil, and a third outermost coil surrounding at least a portion of a length of the second middle coil, wherein the coil assembly comprises a distal end that is located proximal to the distal end of the microcatheter. Page 8

21.-45 (canceled)
